(19) United States
(12) Patent Application Publication
(10) Pub. No.: US 2026/0094487 A1
(43) Pub. Date: Apr. 2, 2026

(54) System, Method, and Device for Prescribing and Dispensing Products

(71) Applicant: Krista Thompson, Orlando, FL (US)

(72) Inventor: Krista Thompson, Orlando, FL (US)

(21) Appl. No.: 19/246,562

(22) Filed: Jun. 23, 2025

Related U.S. Application Data
(60) Provisional application No. 63/702,061, filed on Oct. 1, 2024.

(51) Int. Cl. G07F 17/00; A61B 5/00; A61B 5/024; A61B 5/1171; G16H 20/13; G16H 80/00

(52) U.S. Cl. CPC G07F 17/0092; A61B 5/0077; A61B 5/024; A61B 5/1171; A61B 5/441; A61B 5/7267; A61B 5/749; G16H 20/13; G16H 80/00

(57) ABSTRACT

Embodiments of the present invention comprise a device, system, and method for prescribing and dispensing products. A device may have a graphical user interface and a speaker for providing output to a user. The device may have a camera, microphone, a keyboard, and a diagnostic tester for receiving input from the user. Input from the user may be images of the user that are analyzed by a memory and a processor to determine a suggested product for the user. A user confirmation and/or external confirmation from a licensed medical professional may be provided. Upon receiving a confirmation, the suggested product may be dispensed from the device. Interaction between the user and the device may be aided by the depiction of an avatar on the graphical user interface. The avatar may display expressions that are dynamically updated based on input from the user.

### CROSS-REFERENCE TO RELATED APPLICATIONS

This application claims the benefit of priority of U.S. Provisional Patent Application No. 63/702,061, filed on Oct. 1, 2024, which is hereby incorporated by reference.

### BACKGROUND OF THE INVENTION

Medical products, including prescription and over-the-counter treatments, are typically purchased in-person in a pharmacy setting. When advice is needed before purchasing these treatments, one often needs to seek the advice of a licensed medical professional. Seeking this advice can be inconvenient since one typically needs to schedule an appointment with a medical professional far in advance. Therefore, it is often impossible to obtain necessary advice regarding medical products at the point of purchase.

Vending machines exist to dispense a wide range of products including but not limited to food items, beverages, toiletries, and contraceptives. While vending machines provide a fast shopping experience, there is generally minimal feedback associated with using a vending machine. Someone using a vending machine must know exactly what they want to purchase from the vending machine since vending machines by nature do not have human operators available at the point of purchase.

### SUMMARY OF THE INVENTION

Embodiments of the present invention comprise a device with a graphical user interface (GUI), a keyboard, a camera, a microphone, and a speaker. The keyboard may be used to receive textual input from a user. The microphone may be used to receive audio input from the user. The camera may be used to receive visual input from the user. The graphical user interface may be used to provide visual output to the user. This visual output may be in the form of images, videos, text, and/or numbers. The speaker may be used to provide audio output to the user.

In some embodiments, the keyboard may be integral with the graphical user interface, and therefore may exist as a “touchscreen” as known in the art of electronic devices. The touchscreen may comprise an electrically conductive layer that, when contacted by a user, causes a change in an electrical current flowing through the conductive layer. Certain contact points of the graphical user interface may be associated with human-readable text symbols, and therefore textual input may be provided via the graphical user interface in embodiments wherein the keyboard is integral with the graphical user interface to form a touchscreen.

The device may further comprise a diagnostic tester. The diagnostic tester may be configured to interact with the user to obtain diagnostic data from the user. The diagnostic tester may be a heart rate monitor whereby interacting with the user comprises contacting a location on the user's body and whereby obtaining diagnostic data from the user comprises measuring changes in blood volume in that location on the user's body. In another example, the diagnostic tester may be a scale whereby interacting with the user comprises bearing the weight of the user and whereby obtaining diagnostic data from the user comprises measuring the user's weight. In another example, the diagnostic tester may be the camera of the device whereby interacting with the user and obtaining diagnostic data from the user both comprise capturing one or more images of the user. The diagnostic data obtained from the diagnostic tester may be in the form of machine-readable input.

The device may further comprise a processor and a memory. The memory may comprise a non-transitory computer-readable medium storing instructions that, when executed by the processor, cause the processor to perform a series of steps. Some of the instructions stored in the non-transitory computer-readable medium may be training data. Other instructions stored in the non-transitory computer-readable medium may be instructions for dynamically updating the training data when input is received. This input may be machine-readable input from a user or from an external device. The input may be a rejection of an output provided by the device, thereby causing the training data to be dynamically updated to cause the processor to provide different output in a future situation. The input may alternatively be a confirmation of an output provided by the device, thereby causing the training data to be dynamically updated to strengthen its associated with the output that was confirmed and the input that was received.

The device may further comprise a transmitter and a receiver. The transmitter may be configured to transmit audio, visual, textual, and machine-readable input from the microphone, camera, keyboard, and diagnostic tester, respectively, to an external device. The receiver may be configured to receive visual and textual input from the external device and output the visual and textual input via the graphical user interface. The receiver may be configured to receive audio input from the external device and output the audio input via the speaker. The receiver may be configured to receive machine-readable input from the external device and output the machine-readable input via the processor, whereby the machine-readable input causes the processor to perform a series of steps. The receiver may further output the machine-readable input from the external device to the memory such that the machine-readable input is stored as instructions in the non-transitory computer-readable medium.

The device may further comprise a reservoir that may contain products. The products may be treatments for ailments such as but not limited to creams for treating skin conditions, creams for treating pain, oral medications for treating skin conditions, oral medications for treating erectile disfunction, oral medications for treating pain, oral medication for treating allergies, or nasal sprays for treating allergies. The products may further be cosmetics such as but not limited to mascara, eyeliner, or blush.

A dispenser may eject at least one of the products from the reservoir when the dispenser is activated by the processor. The dispenser may be activated by the processor when the processor is activated by machine-readable input from the device or from an external device. At least one coil of the dispenser may contact the products. The at least one coil may also be connected to at least one motor. When the at least one motor is activated by the processor, the at least one coil may be caused to rotate, thereby causing the products contacting the coil to translate through the reservoir towards a chute of the dispenser and eventually into the chute of the dispenser. When a product is translated into the chute of the dispenser by the at least one coil, the chute may remove the product from the reservoir using gravity. The product may fall to a chute bottom whereby the product is accessible to the user via a product access aperture in the device.

Embodiments of the present invention may also comprise a system. One component of the system may be a device, which may be the same device described previously herein. The device may alternatively be a similar device with a reservoir containing products, a dispenser configured to dispense the products from the reservoir, a graphical user interface, a keyboard, a camera, a microphone, a speaker, a processor, a memory, and a diagnostic tester which in some embodiments may also be the camera.

An external device of the system may be communicatively coupled with the device via a network. A transmitter of the system may be configured to transmit audio, visual, textual, and machine-readable input from the microphone, camera, keyboard, and diagnostic tester, respectively, to an external device. The machine-readable input transmitted by the transmitter may also be transmitted from the memory of the device to the external device. A receiver of the system may be configured to receive visual and textual input from the external device and output the visual and textual input via the graphical user interface. The receiver may be configured to receive audio input from the external device and output the audio input via the speaker. The receiver may be configured to receive machine-readable input from the external device and output the machine-readable input via the processor, whereby the machine-readable input causes the processor to perform a series of steps. The series of steps may include causing the dispenser to eject at least one of the products from the reservoir. The receiver may further output the machine-readable input from the external device to the memory such that the machine-readable input is stored as instructions in the non-transitory computer-readable medium. The transmitter and the receiver may be components of the device itself, or alternatively may be separate components of the system that are electronically connected to the device.

Training data of the system may be stored in the memory. Instructions for dynamically updating the training data may further be stored in the memory. The instructions for dynamically updating the training data may update the training data based on input received from a user via the keyboard, camera, or microphone. The instructions for dynamically updating the training data may also update the training data based on input received from the external device. The input received from the user and external device may be in the form of machine-readable instructions.

The system may further comprise product packaging encasing each product configured in the reservoir of the device. In embodiments wherein the dispenser comprises coils contacting the products, the product packaging may be the components that contact the coils such that the product contained within the product packaging does not directly contact the coils. In some embodiments, the device may comprise a printer configured to print alphanumeric characters on the product packaging. The printer may be activated by the processor to print alphanumeric characters on the product packaging of at least one product. The printer may be activated based on input from the user or the external device.

Embodiments of the present invention may also comprise a method. A device may be provided. The device may be the same device described previously herein. The device may alternatively be a similar device with a reservoir, a dispenser configured to dispense the products from the reservoir, a graphical user interface, a keyboard, a camera, a microphone, a speaker, a processor, a memory, and a diagnostic tester which in some embodiments may also be the camera.

Products may be secured in the reservoir of the device such that the products are only accessible to a user via the dispenser. A first user input may be received from the user via the keyboard, camera, or microphone. In response to the first user input, a first device output may be provided to the user via the graphical user interface and/or speaker. The first device output may be instructions for using the diagnostic tester. The diagnostic tester may be used to receive a second user input. The second user input may be user data, which may be images of the user and/or measurements of various parameters of the user. The measurements of the various parameters of the user may include but are not limited to the user's height, weight, and heartrate. The second user input may be received in the form of machine-readable instructions so that the second user input can be processed by the processor and the memory.

The processor and the memory may be used to determine a second device output based on the second user input. The second device output may be a suggestion for a first product. The second device output may be provided to the user via the graphical user interface and/or speaker. A third user input may be received via the keyboard, camera, or microphone confirming the first product. The dispenser may then be activated by the processor to eject the first product form the reservoir so that the first product is accessible to the user.

Alternatively, the third user input may be a rejection of the first product. In this scenario, the processor and the memory may be used to determine a third device output based on the second user input and the third user input. The third device output may be a suggestion for a second product. The third device output may be provided to the user via the graphical user interface and/or speaker. A fourth user input may be received via the keyboard, camera, or microphone confirming the second product. The dispenser may then be activated by the processor to eject the second product form the reservoir so that the first product is accessible to the user.

Training data may be provided to the memory. The training data may comprise suggestions of products based on various possible user data. Instructions for dynamically updating the training data may also be provided to the memory. When the third user input is a rejection of the first product, the processor may be used to update the training data based on the third user input. The processor may also be used to update the training data based on the third user input and the fourth user input when the third user input and fourth user input are confirmations of the product suggested to the user. In these scenarios, the training data may be updated to further strengthen the association between the suggested products and the measurements or images present in the user data received from the diagnostic tester.

In some embodiments, an external device may be provided. The second device output and third device output may be provided to the external device before the second device output and third device output are provided via the graphical user interface and/or speaker. A first expert input may be received from the external device. The first expert input may be a confirmation or rejection of the first suggested product. The second device output and third device output may not be provided via the graphical user interface and/or speaker until the first expert input provides a confirmation of the suggested product. In some embodiments, the second device output and/or third device output may be provided via the graphical user interface and/or speaker before the first expert input provides a confirmation of the suggested product, but the processor may not be used to activate the dispenser until the first expert input provides a confirmation of the suggested product. The training data may be dynamically updated by the processor using the instructions for dynamically updating the training data when the first expert input is received.

In example embodiments, the diagnostic tester may be the camera. Receiving the second user input may comprise using the camera to obtain an image of the user. The training data may comprise a list of skin disorders, at least one image associated with each of the skin disorders in the list of skin disorders, and at least one product associated with each of the skin disorders in the list of skin disorders. The product(s) suggested by the second device output and third device output may be products used to treat a skin disorder.

In some embodiments, the training data may comprise facial expressions of an avatar. In these embodiments, at least a portion of the first device output and the second device output may comprise an image and/or video of an avatar displayed on the graphical user interface. At least a portion of the first user input, the second user input, the third user input, and the fourth user input may be facial expression of the user received by the camera. At least a portion of the first user input, the second user input, the third user input, and the fourth user input may be tones of voice of the user received by the microphone. The processor may be used to update the training data based on the facial expressions and/or tones of voice in the first user input, second user input, third user input, and/or fourth user input.

In some embodiments, the device may comprise a printer. The printer may be configured to print alphanumeric characters when activated by the processor. A fifth user input may be received via the keyboard, microphone, or camera. The fifth user input may comprise user unique identifier data including but not limited to the user's name, address, and date of birth. The user unique identifier data may be stored in the memory. The processor may be used to activate the printer to print the user unique identifier data on the first product or the packaging of the first product before activating the dispenser to eject the first product from the reservoir. The processor may further be used to activate the printer to print information about the first product such as a brand name, active ingredients, and use directions on the first product or the packaging of the first product before activating the dispenser to eject the first product from the reservoir.

### BRIEF DESCRIPTION OF DRAWINGS

FIG. 1 shows a diagram of a system according to embodiments of the present invention.

FIG. 2 shows a front view of a device according to embodiments of the present invention.

FIG. 3 shows a side cross-section view of the device of FIG. 1.

FIG. 4 shows a flowchart illustrating a method according to embodiments of the present invention.

FIG. 5 shows a flowchart illustrating a method according to embodiments of the present invention.

FIG. 6 shows a flowchart illustrating a method according to embodiments of the present invention.

FIG. 7A shows a front view of a graphical user interface depicting an avatar with a positive expression.

FIG. 7B shows a front view of a graphical user interface depicting an avatar with a neutral expression.

### DETAILED DESCRIPTION OF THE INVENTION

The description provided herein describes example embodiments of the present invention and is not intended to limit the present invention to any particular embodiment, feature, component, material, chemical composition, use, functionality, method steps, or any other property. Furthermore, the drawings provided herein show example embodiments of the present invention and are not intended to limit the present invention to any particular embodiment, feature, component, material, chemical composition, use, functionality, method steps, or any other property.

Throughout this disclosure, the terms “machine-readable data”, “machine-readable instructions”, and “machine-readable input” may be used interchangeably.

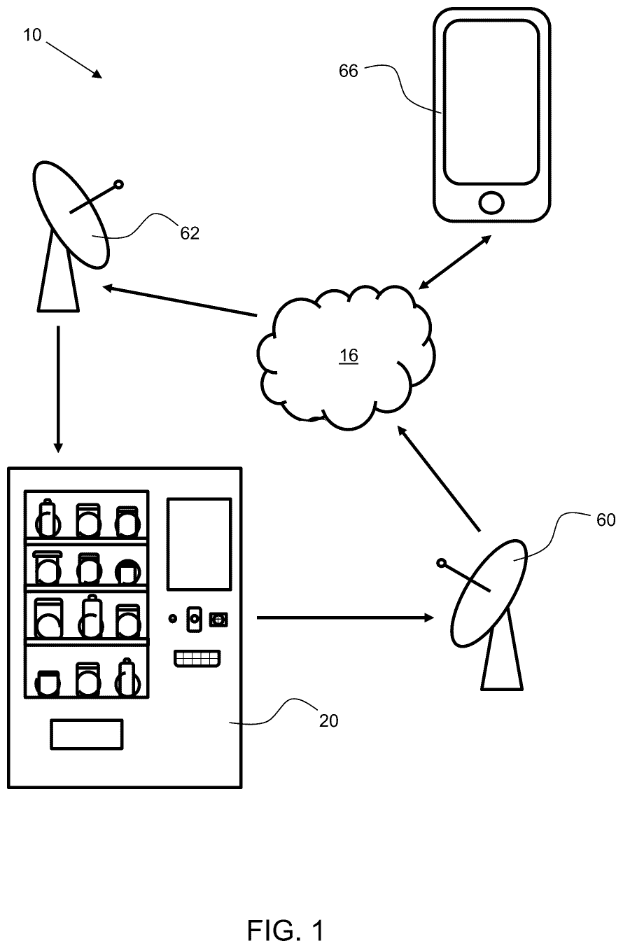

As shown in FIG. 1, a system 10 comprises a device 20 and an external device 66 connected to one another via a network 16. The network 16 may be a wired network such as but not limited to an ethernet network, local area network (LAN), personal area network (PAN), or a public switched telephone network (PSTN). Alternatively, the network 16 may be a wireless network such as but not limited to the internet, a Bluetooth connection, or a radio network. The device 20 may be a device for dispensing products as described further herein. The external device 66 may be any device capable of receiving, processing, and outputting machine-readable data. The external device 66 may further be a device capable of forming a connection to the network 66 via a wired or wireless connection. Examples of the external device 66 include but are not limited to a smartphone, smart watch, tablet, personal computer, or laptop computer.

A transmitter 60 may be used to transmit information from the device 20 to the network 16. This information may be in the form of machine-readable data, human-readable images, and/or sound saves. The transmitter 60 may be incorporated into the device 20 or alternatively may be a separate component of the system 10. A receiver 62 may be used to receive information from the network 16 and transfer said information to the device 20. This information may be in the form of machine-readable data, human-readable images, and/or sound saves. The receiver 62 may be incorporated into the device 20 or alternatively may be a separate component of the system 10. The external device 66 may comprise its own transmitter and receiver for transmitting and receiving information to and from the network 16.

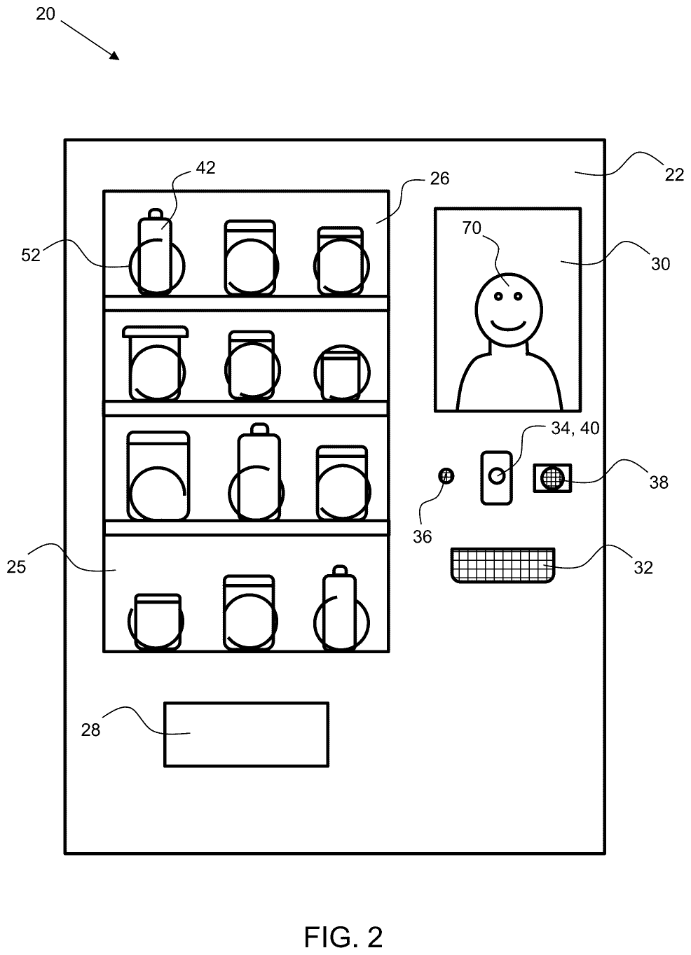

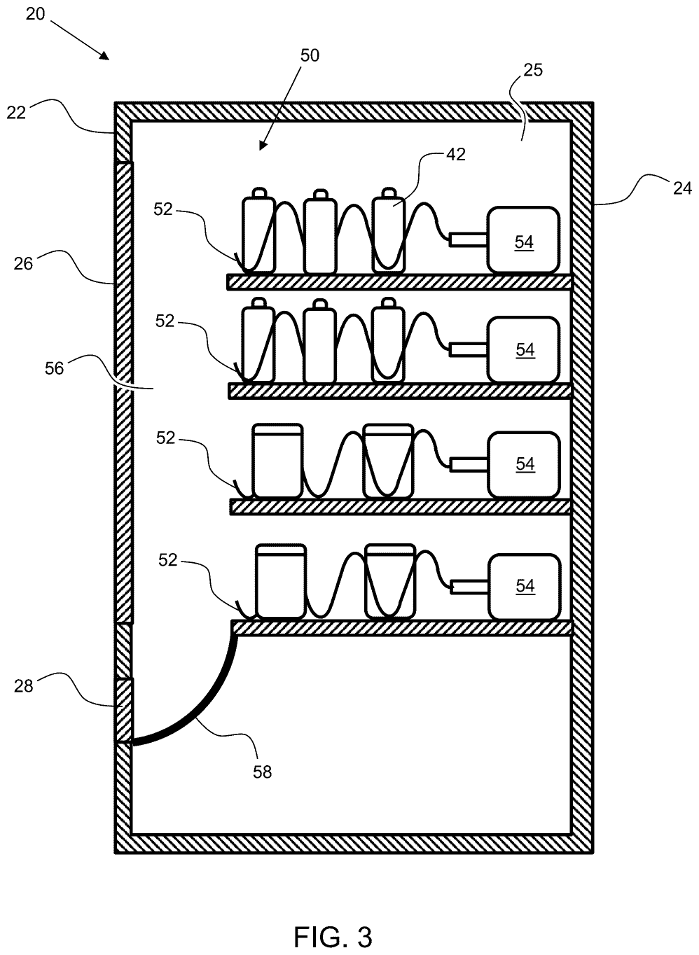

As shown in FIGS. 2-3, a device 20 has a reservoir 25 that contains products 42. The products 42 are visible from a device front 22 through a product window 26. Also visible from the device front 22 is a graphical user interface 30 that may be configured to display an avatar 70. A keyboard 32, camera 34, microphone 36, and speaker 38 are configured at the device front 22. A diagnostic tester 40, which may be the camera 34 in some embodiments of the present invention, is also configured at the device front 22.

The keyboard 32 is used by a user to provide input to the device 20 in the form of human-readable text, into which a processor of the device may translate into machine-readable data. The camera 34 is used by a user to provide input to the device 20 in the form of human-readable images, into which the processor of the device may translate into machine-readable data. The microphone 36 is used by a user to provide input to the device 20 in the form of sound waves, into which the processor of the device 20 may translate into machine-readable data. The speaker 38 may be used to transmit output from the device 20 to the user. The output may be translated by the processor of the device 20 from machine-readable data into electronic signals capable of causing the speaker 38 to produce sound waves.

Using the keyboard 32, camera 34, and microphone 36, the user may interact with the avatar 70 displayed on the graphical user interface 30. The avatar 70 may provide visual output to the user in the form of human-readable images. In some embodiments, human-readable images other than the avatar 70 may be displayed on the graphical user interface 30. These other human-readable images may be but are not limited to alphabetic characters, numerical characters, or symbols. The information displayed on the graphical user interface 30 may be used to instruct the user on using the diagnostic tester 40. The diagnostic tester 40 may be used to obtain information from the user such as but not limited to the appearance of the user's skin, the user's heart rate, the user's height, the user's weight, or the user's blood pressure. The information displayed on the graphical user interface 30 may be aided by information provided through the speaker 38 in the form of sound waves. For example, text stating “Approach the camera” may be displayed on the graphical user interface 30 at the same time as spoken words stating “Approach the camera” are outputted via the speaker 38.

The products 42 held in the reservoir 25 of the device 20 may be ejected from the reservoir 25 by a dispenser 50. The reservoir 25 is configured between the device front 22 and a device rear 24. The dispenser may comprise coils 52 configured around the products 42 within the reservoir 25. Each coil 52 is connected to a motor 54 that, when activated, causes its respective coil 52 to rotate. Rotation of a coil 52 causes the products 42 around which the coil 52 is configured to translate towards a chute 56 of the dispenser 50. The chute 56 may be an area between the coils 52 and the device front 22. The products 42 may be translated past the coils 52 such that they enter the chute 56 and fall down the chute 56 due to the force of gravity acting on the products 42. A product access aperture 28 at the device front 22 allows a user to access a chute bottom 58 where the products 42 come to rest after falling down the chute 56.

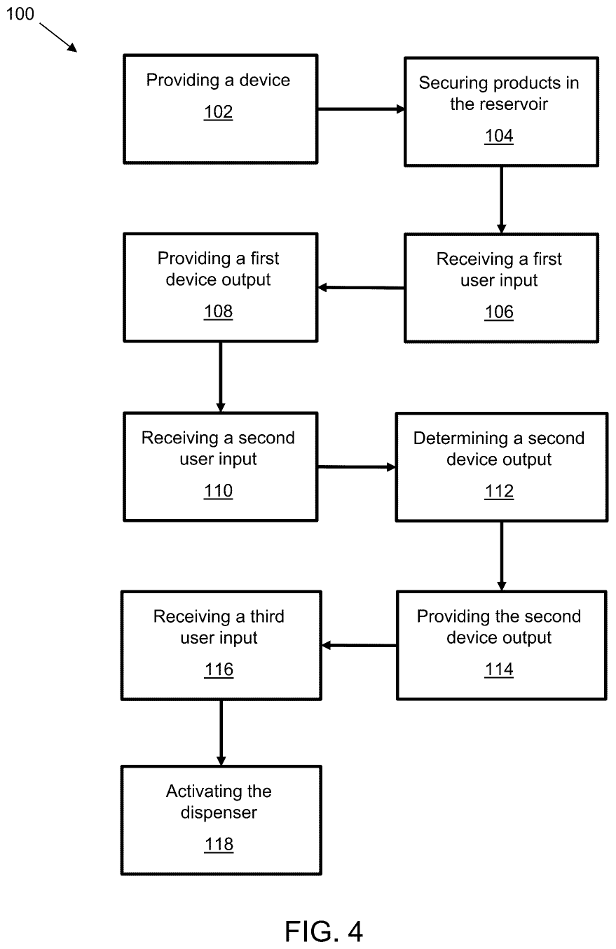

As shown in FIG. 4, a method 100 comprises first providing a device 102. The device may be the device 20 shown and described in FIGS. 2-3. Products may be secured in the reservoir of the device 104. A first user input may be received 106. The first user input may be a textual input via the keyboard of the device, an audio input via the microphone of the device, or a visual input via the camera of the device. In some embodiments, the camera of the device may detect a user approaching the device and may receive the first user input as a visual input of the user. In other embodiments, the first user input may be a detailed description of an ailment the user is experiencing or a description of a diagnostic test the user would like to have performed.

A first device output may be provided 108 to the user. The first device output may be a welcome message provided via the graphical user interface of the device and/or via the speaker of the device. The first device output may instruct the user on using the diagnostic tester of the device. The first device output may be determined using the first user input, a processor of the device, and a memory of the device.

The diagnostic tester may be used to receive a second user input 110. The second user input may be information about the user obtained from the diagnostic tester. In some embodiments, this information may be an image of the user's face obtained using the camera of the device. In these embodiments, the camera of the device may serve as the diagnostic tester. The information about the user may be used to determine a second device output 112. The second device output may be determined by analyzing the information about the user using the processor and the memory of the device. The memory of the device may contain associations between images of skin conditions, names of associated skin conditions, and treatments for associated skin conditions.

Part of the second user input may also be a detailed description of an ailment the user is experiencing. For example, receiving the second user input 110 may comprise obtaining an image of the user's face using the camera and receiving a written description from the user stating that they have red spots only on their face. Determining the second device output 112 may then comprise the processor analyzing the image of the user's face and associating a number of skin conditions that result in red spots on the face such as rosacea, eczema, lupus, and acne. The processor may determine that acne is not an appropriate associated skin conditions given the lack of black or white spots surrounded by red spots in the image(s) received by the camera. The processor may further pair the information provided by the user via the keyboard stating that the red spots are only on the user's face with the visual information from the camera and determine that the associated skin condition cannot be eczema or lupus since the red spots only appear on the user's face and not elsewhere on the user's body. Therefore, the processor may determine that the associated skin condition is rosacea. Determining the second device output 112 may then comprise the processor searching the memory for an associated treatment for rosacea.

In another example, receiving the second user input 110 may comprise obtaining an image of the user's face using the camera and receiving a written description from the user stating that they have dark spots on their face. Determining the second device output 112 may then comprise the processor analyzing the image of the user's face and associating a number of skin conditions that result in dark spots on the face such as sun damage and hyperpigmentation. At this point, additional device outputs and user inputs may be exchanged between the user and the device that are not shown in the flowchart in FIG. 1. For example, a question in the form of human-readable text may appear on the graphical user interface of the device asking if the user has experienced recent prolonged exposure to the sun. The user may answer the question by providing textual input via the keyboard or audio input via the microphone. The user may provide input stating that they have not experienced recent prolonged exposure to the sun. The processor may then use this additional user input to determine that the associated skin condition is hyperpigmentation. Determining the second device output 112 may then comprise the processor searching the memory for an associated treatment for hyperpigmentation.

After determining the second device output 112, the second device output is provided 114 to the user via the graphical user interface and/or speaker of the device. The second device output may be a determination of an associated skin condition and a suggested associated treatment. A third user input may be received 116 confirming the associated treatment. The dispenser of the device may then be activated 118 by the processor of the device to dispense a product, wherein the product is the suggested associated treatment confirmed by the user via the third user input.

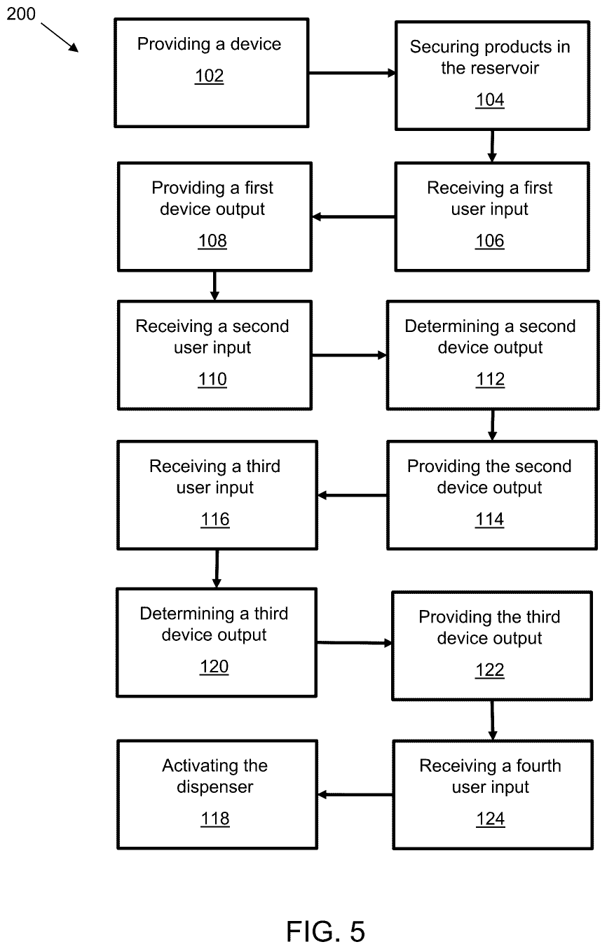

As shown in FIG. 5, the third user input may not be a confirmation of the suggested treatment but rather may be a denial of the suggested treatment. In these cases, a third device output 120 may be determined using the information previously stored in the memory of the device along with the information provided as the third user input. As part of receiving the third user input 116, the user may provide additional information about their skin condition using the keyboard, camera, or microphone. For example, determining the second device output 112 may comprise determining that the user experiences a chronic skin condition due to red marks on their face. However, receiving the third user input 116 may comprise the user providing information via the keyboard or microphone stating that they recently had prolonged exposure to the sun without sun protection and believe the red marks on their face are sunburn and not a chronic skin condition. In some embodiments, the memory of the device may then be updated so that additional device outputs are provided to future users asking said users if they have experienced prolonged exposure to the sun without sun protection if red marks are noticed on said users' faces when receiving the second user input 110 from said users.

After determining the third device output 120, the third device output is provided 122 to the user via the graphical user interface and/or speaker of the device. The third device output may be a determination of an associated skin condition and a suggested associated treatment. A fourth user input may be received 124 confirming the associated treatment. The dispenser of the device may then be activated 118 by the processor of the device to dispense a product, wherein the product is the suggested associated treatment confirmed by the user via the fourth user input.

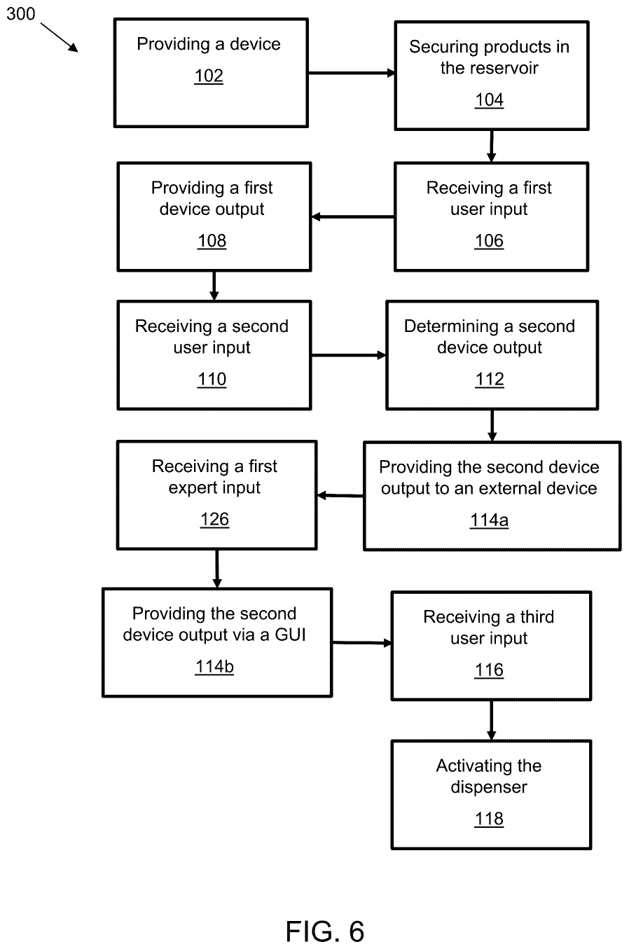

As shown in FIG. 6, the second device output may be provided to an external device 114a after the second device output is determined 112. The external device may be any device capable of receiving, processing, and outputting machine-readable data. The external device may further be a device capable of forming a connection to a network via a wired or wireless connection. Examples of the external device include but are not limited to a smartphone, smart watch, tablet, personal computer, or laptop computer. A licensed medical professional may use the external device to provide a first expert input wherein the first expert input is received 126 by the device. The first expert input may be a prescription for a product contained within the device if regulations require that a prescription be issued to provide said product to a user. The second device output may then be provided to the user via the graphical user interface of the device 114b. The second device output may be the prescription for the product. A third user input may be received 116 confirming the prescribed product. The dispenser of the device may then be activated 118 by the processor of the device to dispense the prescribed product.

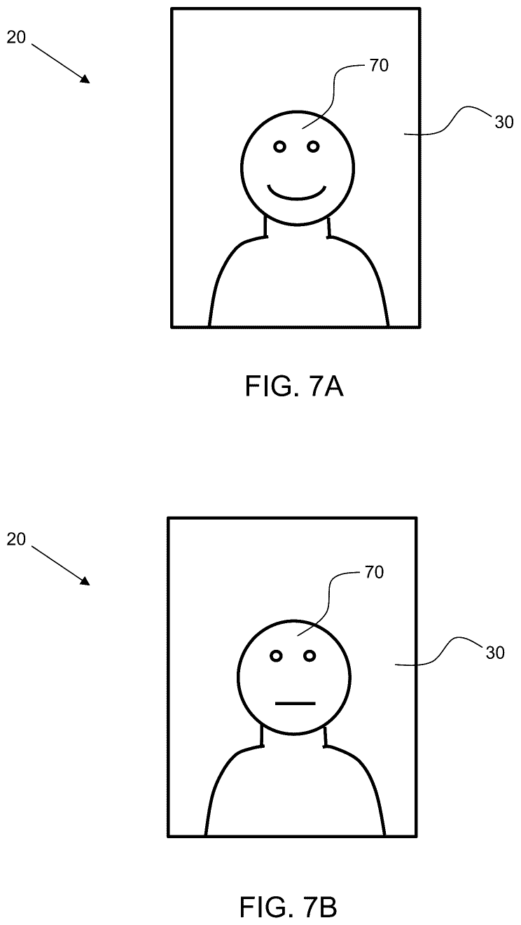

As shown in FIGS. 7A-7B, an avatar 70 is displayed on the graphical user interface 30 of a device 20. The avatar may be displayed as an electronic image of a person or a series of electronic images of a person shown consecutively to portray motion of the avatar 70 on the graphical user interface 30. As shown in FIG. 7A, the avatar 70 may display a positive expression when a user first approaches the device 20 and provides a first user input. The device may use a camera and/or microphone to obtain visual and audio input from the user. A processor of the device, in conjunction with a memory of the device, may use the visual and audio input from the user to determine an emotional state of the user. Training data may be provided to the memory. The training data may comprise a list of facial expressions, a list of voice tones, and a list of emotional states. Each of the emotional states in the list of emotional states may be associated with one or more of the facial expressions from the list of facial expressions and one or more voice tones in the list of voice tones. When receiving user input via the camera and microphone, the processor, in conjunction with the memory, may associate the visual input with one or more facial expressions from the list of facial expressions. The processor, in conjunction with the memory, may also associate the audio input with one or more voice tones from the list of voice tones. The processor, in conjunction with the memory, may then associate the associated facial expression(s) and the associated voice tone(s) with an emotional state of the user.

The avatar 70 may retain its positive expression as shown in FIG. 7A or may adopt a neutral expression as shown in FIG. 7B depending on the emotional state of the user. For example, if the emotional state of the user is determined to be happiness, the avatar 70 may retain its positive expression. If the emotional state of the user is determined to be fear, the avatar 70 may still retain its positive expression in an attempt to make the user feel more comfortable. However, if the emotional state of the user is determined to be anger or frustration, the avatar 70 may adopt a neutral expression as to not further anger or frustrate the user. The training data of the memory may comprise a list of facial expressions of the avatar 70. Each of the facial expressions of the avatar 70 may be associated with one or more of the emotional states in the list of emotional states.

The avatar 70 may change its facial expression at any time throughout an interaction with a user; not just after receiving the first user input. Furthermore, the training data may be dynamically updated to alter the associations between facial expression of the avatar and the emotional states of the user, between the facial expressions and the emotional states of the user, and between the voice tones and the emotional states of the user. The training data may be dynamically updated in this manner upon receiving feedback from the user. For example, if the avatar 70 adopts a positive expression due to a perceived positive first expression from the user and the user then adopts a negative second expression, the training data may be dynamically updated to associate the first expression of the user with a negative emotional state, and therefore cause the avatar 70 to adopt a neutral expression when future users express the same first expression.

1. A device comprising:
a graphical user interface;
a keyboard;
a camera;
a microphone;
a speaker;
a processor;
a memory comprising a non-transitory computer-readable medium storing instructions that, when executed by the processor, cause the processor to perform a series of steps;
a diagnostic tester configured to interact with a user and thereby obtain diagnostic data from the user;
a transmitter configured to transmit audio, visual, textual, and machine-readable input from the microphone, camera, keyboard, and diagnostic tester, respectively, to an external device;
a receiver configured to receive audio, visual, textual, and machine-readable input from the external device and provide output via the speaker, graphical user interface, and processor;
a reservoir containing products; and
a dispenser that, when activated by the processor, ejects at least one of the products from the reservoir.

2. The device of claim 1, wherein the diagnostic tester is the camera.

3. The device of claim 1, wherein the instructions stored in the memory comprise:
training data; and
instructions for updating the training data when machine-readable input is received from the user or the external device.

4. The device of claim 1, wherein the processor is activated by machine-readable input from the external device to activate the dispenser.

5. The device of claim 1, wherein the dispenser comprises a chute that removes products from the reservoir using gravity.

6. The device of claim 5, wherein the dispenser comprises:
at least one coil contacting the products;
at least one motor, that when activated by the processor, causes the at least one coil to rotate, thereby causing at least one of the products to translate along the at least one coil towards the chute,
whereby the at least one of the products is configured within the chute and is ejected via the chute.

7. The device of claim 1, wherein the keyboard is integral with the graphical user interface.

8. A system comprising:
a device comprising:
a reservoir;
a dispenser;
a graphical user interface;
a keyboard;
a camera;
a microphone;
a speaker;
a processor;
a memory comprising a non-transitory computer-readable medium storing instructions that, when executed by the processor, cause the processor to perform a series of steps;
a diagnostic tester;
an external device communicably coupled with the device via a network;
products configured within the reservoir of the device;
a transmitter configured to transmit audio, visual, textual, and machine-readable input from the microphone, camera, keyboard, and diagnostic tester of the device, respectively, to the external device via the network; and
a receiver configured to receive audio, visual, textual, and machine-readable input from the external device via the network and provide output via the speaker, graphical user interface, and processor of the device,
wherein output provided to the processor of the device causes the dispenser to eject at least one of the products from the reservoir.

9. The system of claim 8, further comprising product packaging encasing each product,
wherein the device further comprises a printer configured to print alphanumeric characters on the product packaging of at least one product when the printer is activated by the processor.

10. The system of claim 8, further comprising:
training data stored in the memory; and
instructions for updating the training data when machine-readable input is received from the user or the external device, the instructions for updating the training data being stored in the memory.

11. A method comprising:
providing a device comprising:
a reservoir;
a dispenser;
a graphical user interface;
a keyboard;
a camera;
a microphone;
a speaker;
a processor;
a memory comprising a non-transitory computer-readable medium storing instructions that, when executed by the processor, cause the processor to perform a series of steps;
a diagnostic tester;
securing products in the reservoir whereby the products are only accessible to a user via the dispenser;
receiving a first user input from a user via the keyboard, camera, or microphone;
providing a first device output to the user via the graphical user interface or speaker, the first device output comprising instructions for using the diagnostic tester;
receiving a second user input from the user via the diagnostic tester, the second user input comprising user data in the form of machine-readable instructions;
using the processor and the memory to determine a second device output comprising a suggestion for a first product;
providing the second device output to the user via the graphical user interface or speaker;
receiving a third user input via the keyboard, camera, or microphone, the third user input comprising a confirmation of the first product; and
activating the dispenser with the processor to eject the first product from the reservoir to be accessible to the user.

12. The method of claim 11, further comprising:
providing the memory with training data, the training data comprising suggestions of products based on various possible user data;
receiving the third user input via the keyboard, camera, or microphone, the third user input comprising a rejection of the first product;
using the processor to update the training data based on the third user input;
using the processor and the memory to determine a third device output comprising a suggestion for a second product;
receiving a fourth user input via the keyboard, camera, or microphone, the fourth user input comprising a confirmation of the second product;
using the processor to update the training data based on the fourth user input; and
activating the dispenser with the processor to eject the second product from the reservoir to be accessible to the user.

13. The method of claim 11, further comprising:
providing an external device;
transmitting the second device output to the external device before providing the second device output via the graphical user interface or speaker;
receiving a first expert input from the external device, the first expert input comprising a confirmation of the first product; and
providing the second device output via the graphical user interface or speaker.

14. The method of claim 11, wherein the diagnostic tester is the camera,
and wherein receiving the second user input comprises using the camera to obtain an image of a user.

15. The method of claim 12, wherein the diagnostic tester is the camera,
wherein receiving the second user input comprises using the camera to obtain an image of the user,
wherein the training data comprises:
a list of skin disorders;
at least one image associated with each of the skin disorders in the list of skin disorders; and
at least one product associated with each of the skin disorders in the list of skin disorders,
and wherein the first product suggested by the second device output is a product used to treat a skin disorder.

16. The method of claim 11, wherein the diagnostic tester is a heart rate monitor,
and wherein receiving the second user input comprises using the heart rate monitor to measure the heart rate of a user.

17. The method of claim 11, further comprising:
providing the memory with training data, the training data comprising facial expressions of an avatar, wherein at least a portion of the first device output and the second device output is a video of the avatar displayed on the graphical user interface; and
using the processor to update the training data based on the first user input, second user input, and third user input.

18. The method of claim 17, wherein at least a portion of each the first user input, second user input, and third user input are facial expressions of a user received by the camera.

19. The method of claim 17, wherein at least a portion of each the first user input, second user input, and third user input are tones of voice of a user received by the microphone.

20. The method of claim 11, wherein the device further comprises a printer configured to print alphanumeric characters when activated by the processor, the method further comprising:
receiving a fifth user input via the keyboard, microphone, or camera, the fifth user input comprising user unique identifier data;
storing the user unique identifier data in the memory; and
using the processor to activate the printer to print the user unique identifier data on the first product before activating the dispenser to eject the first product from the reservoir to be accessible to the user.
